(19) United States
(12) Patent Application Publication
(10) Pub. No.: US 2010/0217128 A1
(43) Pub. Date: Aug. 26, 2010

(54) MEDICAL DIAGNOSTIC DEVICE USER INTERFACE

(76) Inventor: Nicholas Michael Betts, Thebarton (AU)

Correspondence Address: Intellectual Property Dept.;Dewitt Ross & Stevens SC, 2 East Mifflin Street, Suite 600, Madison, WI, 53703-2865, US

(21) Appl. No.: 12/682,088

(22) PCT Filed: Oct. 16, 2008
(86) PCT No.: PCT/AU2008/001525

§ 371 Date: Apr. 8, 2010

(30) Foreign Application Priority Data
Oct. 16, 2007 (AU) 2007905654

(51) Int. Cl. A61B 8/00; G06F 3/041; G06F 3/033; G09G 5/08; G06F 3/048

(52) U.S. Cl. 600/459; 345/173; 345/184; 345/157; 715/835

(57) ABSTRACT

A user interface for a hand held medical diagnostic scanner system, including a display screen and a control member adapted to sense first user control movements in at least one direction and to communicate this movement to software adapted to translate this movement into highlighting of an option indicator, or selection of an option indicator, from a plurality of option indicators displayed on the display screen, the control member being further adapted to sense a second different user control movement and to communicate this movement to software adapted to translate this movement to cause the activation of an option associated with a highlighted option indicator.

### TECHNICAL FIELD

The present invention relates to a user interface for a hand held medical diagnostic ultrasound system, and a method of control of such a hand held medical diagnostic ultrasound system.

### BACKGROUND ART

Ultrasound was first investigated as a medical diagnostic imaging tool in the 1940's. This was based on the use of A-mode (amplitude mode) ultrasound, which is a form of echo ranging. This simply gives a plot of returned echo intensity against time, which, by knowing the speed of sound in the target media, gives the distance of the features returning the echo from the transducer. In order to obtain valid information from such a plot it is necessary that the direction of the transmitted ultrasound beam be constant and known.

In order to provide an imaging system, it is necessary to insonify a larger area, at least a two dimensional slice of the target. It is also necessary to receive returned echoes from this area and to display this information in correct spatial relationship.

Since the only information received by an ultrasound transducer is echo intensity over time, spatial information can most easily be added by knowing the direction from which the echo was received. This means knowing the position and orientation of the transducer at all times and this was most easily achieved by controlling the movement of the transducer.

This led to B-mode (brightness mode) scanning, where the ultrasound output is pulsed and the transducer is mechanically scanned over the target. The transducer detects the echo from each pulse as intensity versus time, called a scanline. The scanlines are displayed with brightness being proportional to echo intensity, thus forming an image.

Ultrasound technology developed significantly in the 1960's with the development of articulated arm B-mode scanners. Articulated arm scanners, also known as static mode scanners, connect the ultrasonic transducer to a moveable arm, with movement of the arm mechanically measured using potentiometers. The articulated arm also ensures that the degree of freedom of movement of the transducer is limited to a defined plane. This allowed the position of the transducer to be known with considerable accuracy, thus allowing the scanlines recorded by the transducer to be accurately located in space relative to each other for display.

Static mode ultrasound scanners were in wide use until the early 1980s. The static mode scanners were large cumbersome devices, and the techniques used are not readily suited to a handheld ultrasound system. These devices would usually be placed in a room dedicated to their use.

The electronic control of these devices was very limited. The returned echo signals were received by a transducer and translated into analogue electrical signals. Amplification and some basic analogue signal processing were applied, and the result displayed on an oscilloscope. The controls were the direct analogue controls of the individual components. No centralised control centre was provided or needed.

In the mid 1970's real-time scanners were developed where an ultrasonic transducer was rotated using a motor. Motor driven transducers removed the need for precise knowledge of the position of the transducer housing, since the operator needed only to hold the transducer housing still and the motor would sweep the transducer rapidly to produce a scan arc. This resulted in an evenly distributed set of scanlines, in a single plane, whose spatial relationship was known because the sweep characteristics were known.

The removal of the articulated arm made these devices smaller and more convenient, but they were still of a size which required a space dedicated to their use.

The rotation motor was superseded by electronic beam steering transducers consisting of a number of electronic crystals where the transmitting pulse can be delayed in sequence to each crystal and thus effect an electronic means to steer the ultrasound beam. The basic technique is still in wide use today, with nearly all modern medical ultrasound equipment using an array of ultrasonic crystals in the transducer.

Electronic beam steering removes the need for a motor to produce real time images. The scanlines resulting from the use of an array transducer are contained within a defined plane, or in the case of 2-D arrays within a defined series of planes. The scanlines may therefore be readily mapped onto a flat screen for display.

The progress of electronics and information technology has made the control of such machines a much more significant problem. The received signal is digitised immediately at the transducer output. This enables a wide range of complex digital signal processing techniques to be applied, which would have been prohibitively difficult to apply in analogue componentry. The user or manufacturer controls these functions by computer program control. The number and range of these controls accessible to a user is considerable. A quite rich and complex user interface is required to manage the range of options.

These machines also provide many convenient features beyond the mere receipt and display of ultrasound scan images. A full computer user interface is provided, enabling patient data to be added and associated with scans. Scans can be stored for later retrieval, or transmitted over computer networks. Text and graphic annotations can be added to scans.

These devices may be semi-portable, no longer requiring a dedicated space, but being mounted on a cart which can be moved to a patient's bedside.

This has greatly extended the usefulness of ultrasound, especially bringing it into the emergency ward and to assist in such routine procedures as line insertion.

However, in order to implement the complex user interface, a keyboard and computer style screen are provided. The need to provide this style of physical user interface is a major contributor to the size and cost of ultrasound units, and represents a requirement which is difficult to further minimise.

### DISCLOSURE OF THE INVENTION

In one form of this invention there is proposed a user interface for a hand held medical diagnostic scanner system, including a display screen and a control member adapted to sense first user control movements in at least one direction and to communicate this movement to software adapted to translate this movement into highlighting of an option indicator, or selection of an option indicator, from a plurality of option indicators displayed on the display screen, the control member being further adapted to sense a second user control movement, different to the first user control movement and to communicate this movement to software adapted to translate this movement to cause the activation of an option associated with a highlighted option indicator.

Preferably, the control member is a thumbwheel or scrollwheel, adapted to be rotated in a forward and a reverse direction, and to be pressed.

In preference, the hand held diagnostic medical scanner is an ultrasound scanner.

In an embodiment, the display screen is a touch screen, and the control member is a portion of that screen, adapted to detect a user's digit being slid in at least one direction, and to detect a tap as the second user control movement.

### BRIEF DESCRIPTION OF THE DRAWINGS

FIG. 1 is a view of a handheld ultrasound scanning device including a preferred embodiment of the present invention;

FIG. 2 is a view of the scanning device shown in FIG. 1 in use.

FIG. 3 is a block diagram of the electronic components of the scanning device of FIG. 1.

FIG. 4 is a view of the home screen of an embodiment of the interface of the invention.

FIG. 5 is a view of an ultrasound application display screen of the interface of the invention.

FIG. 6 is a view of a pan and zoom control screen of the interface of the invention.

FIG. 7 is a view of a measurement management screen of the interface of the invention.

FIG. 8 is a view of a screen of the invention showing a calliper measurement.

FIG. 9 is a view of a screen of the invention showing a polygon measurement.

FIG. 10 is a view of a screen of the invention showing a polygon measurement.

FIG. 11 shows the Image/Exam Scroller function of the interface in use.

FIG. 12 shows the Exam Scroller function of the interface in use.

FIG. 13 shows a screen of the interface of the invention for text annotation.

FIG. 14 shows a screen of the interface of the invention for voice annotation.

### BEST MODE FOR CARRYING OUT THE INVENTION

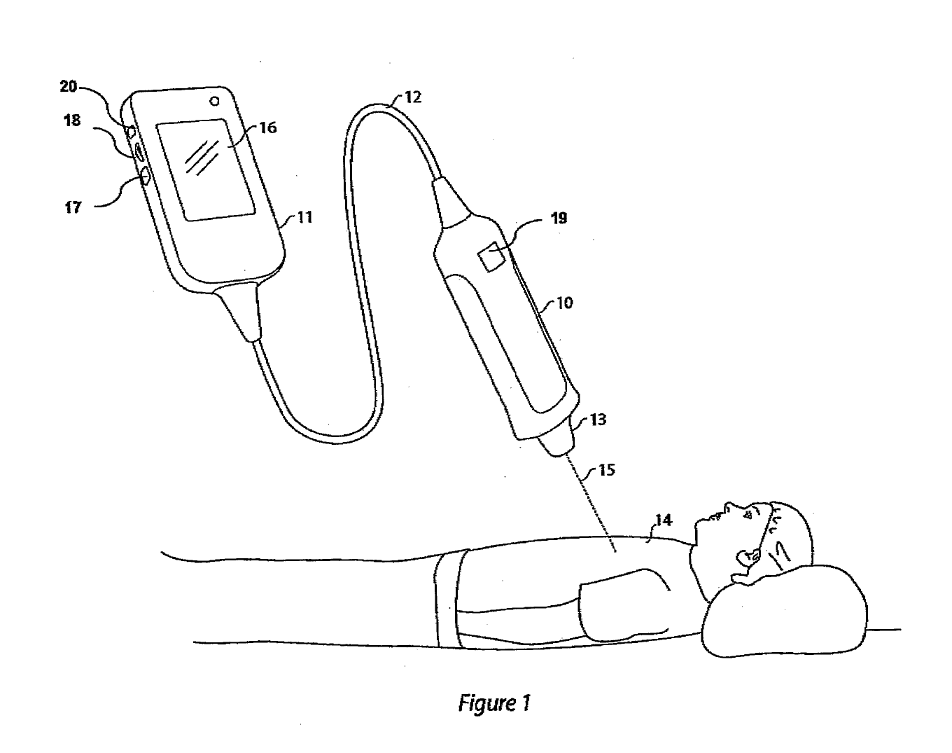

Referring now to FIG. 1, there is illustrated a hand held ultrasound scanning device according to an embodiment of the invention. There is a hand held ultrasonic probe unit 10, a display and processing unit (DPU) 11 with a display screen 15 and a cable 12 connecting the probe unit to the DPU 11. The DPU includes a thumbwheel 18, which is able to be rotated up and down and to be pressed inward to the body of the DPU. These movements provide control signals for the user interface. There is also provided two further interface control buttons, the back button 17, and the start button 20.

The probe unit 10 includes an ultrasonic transducer 13 adapted to transmit pulsed ultrasonic signals into a target body 14 and to receive returned echoes from the target body 14.

In this embodiment, the transducer is adapted to transmit and receive in only a single direction at a fixed orientation to the probe unit, producing data for a single scanline 15.

The probe unit further includes an orientation sensor 19 capable of sensing orientation or relative orientation about one or more axes of the probe unit.

Thus, in general, the sensor is able to sense rotation about any or all of the axes of the probe unit.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

Since the distance between the mounting point of the sensor 19 and the tip of the transducer 13 is known, it would also be possible to implement the sensor with one, two or three accelerometers.

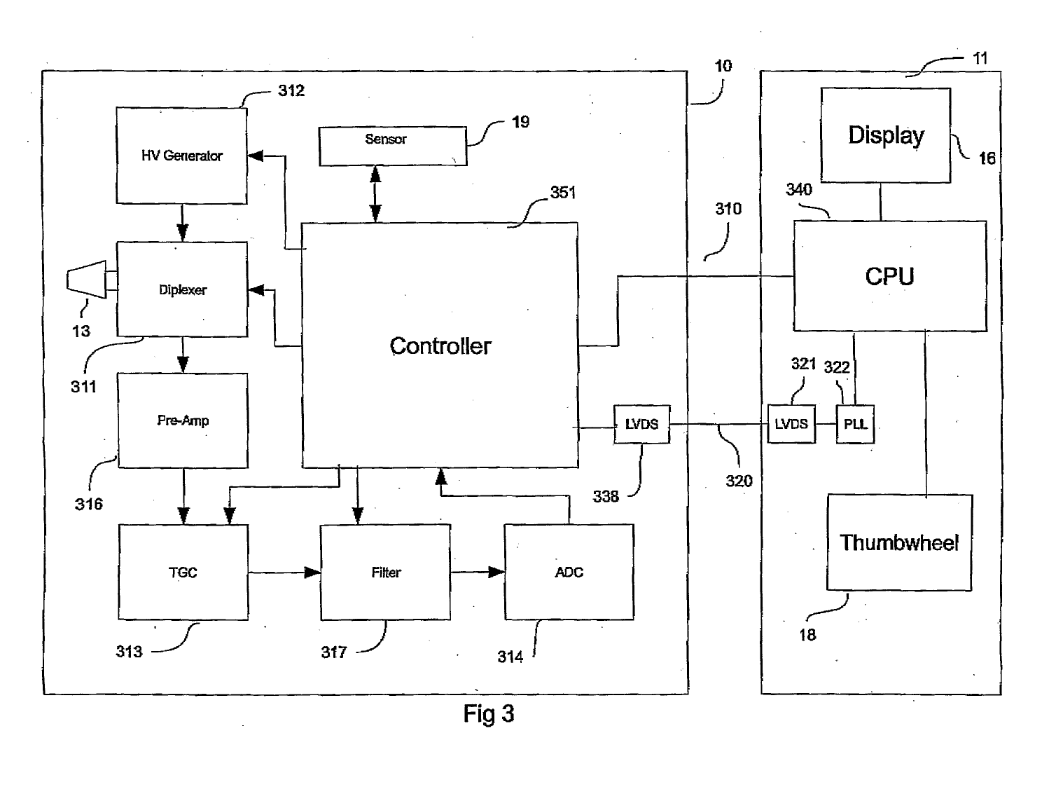

A block diagram of the ultrasonic scan system is shown in FIG. 3. There is a probe unit 10 and a DPU 11. The probe unit includes a controller 351 which controls all of the functions of the probe.

The DPU includes a main CPU 340.

The probe unit 10 communicates with the DPU 11 via a low speed message channel 310 and a high speed data channel 320. The message channel is a low power, always on connection.

The data channel is a higher speed and hence higher power consumption bus which is on only when required to transmit data from the probe unit to the DPU.

The probe unit includes a transducer 13 which acts to transmit and receive ultrasonic signals. A diplexer 311 is used to switch the transducer between transmit and receive circuitry.

On the transmit side the diplexer is connected to high voltage generator 312, which is controlled by controller 351 to provide a pulsed voltage to the transducer 13. The transducer produces an interrogatory ultrasonic pulse in response to each electrical pulse.

This interrogatory pulse travels into the body and is reflected from the features of the body to be imaged as an ultrasonic response signal. This response signal is received by the transducer and converted into an electrical received signal.

The depth from which the echo is received can be determined by the time delay between transmission and reception, with echoes from deeper features being received after a longer delay. Since the ultrasound signal attenuates in tissue, the signal from deeper features will be relatively weaker than that from shallower features.

The diplexer 311 connects the electrical receive signal to time gain compensation circuit (TGC) 313 via a pre-amp 316. The TGC applies amplification to the received signal. The characteristics of the amplification are selected to compensate for the depth attenuation, giving a compensated receive signal where the intensity is proportional to the reflectiveness of the feature which caused the echo. In general, the amplification characteristics may take any shape.

This compensated signal is passed to an analogue to digital converter (ADC) 314, via an anti-aliasing filter 317. The output of the ADC is a digital data stream representing the intensity of the received echoes over time for a single ultrasonic pulse.

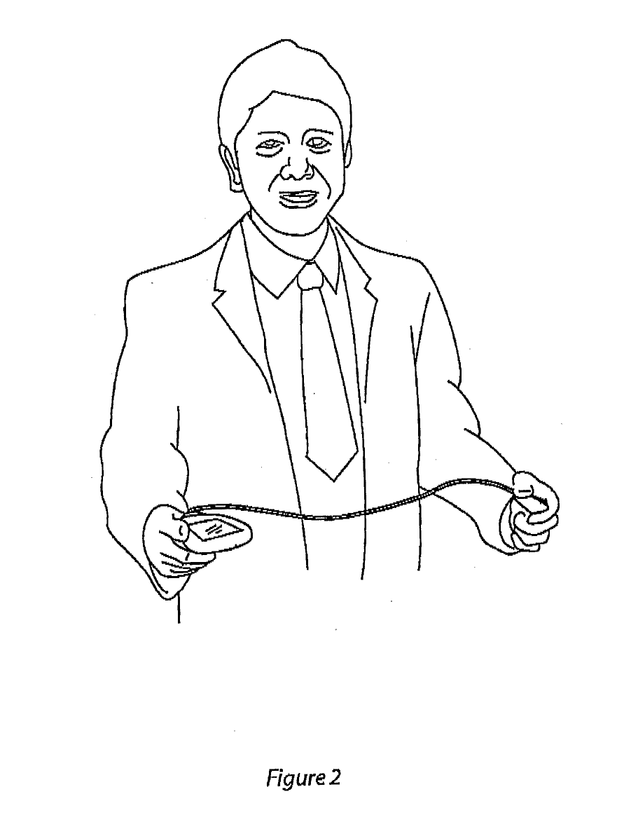

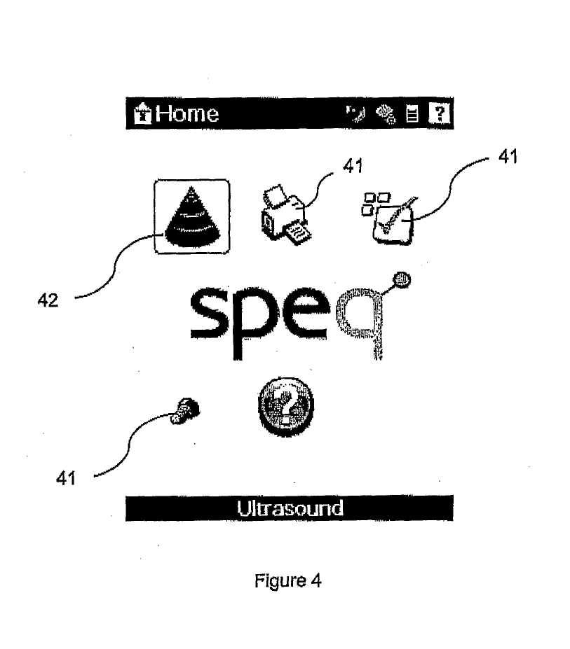

In use, a user applies the probe unit 10 to a body to be imaged 14. To use the scanner, a user may hold the DPU in one hand and the probe unit in the other, as shown in FIG. 2. The display unit displays a home screen 40 as shown in FIG. 4 which displays a number of icons 41. Each icon is associated with a particular function of the scanner device. The appearance of the icon acts as a memory aid to the function. The name of the function highlighted is also displayed at the bottom of the screen.

The user rotates the thumbwheel 18 to sequentially highlight each icon. Pressing the thumbwheel selects the highlighted icon and activates the associated function. The display 16 is a touchscreen. The required icon may also be selected by a user by touching the screen where the icon is displayed.

When a function is selected, the DPU runs an application program associated with that icon.

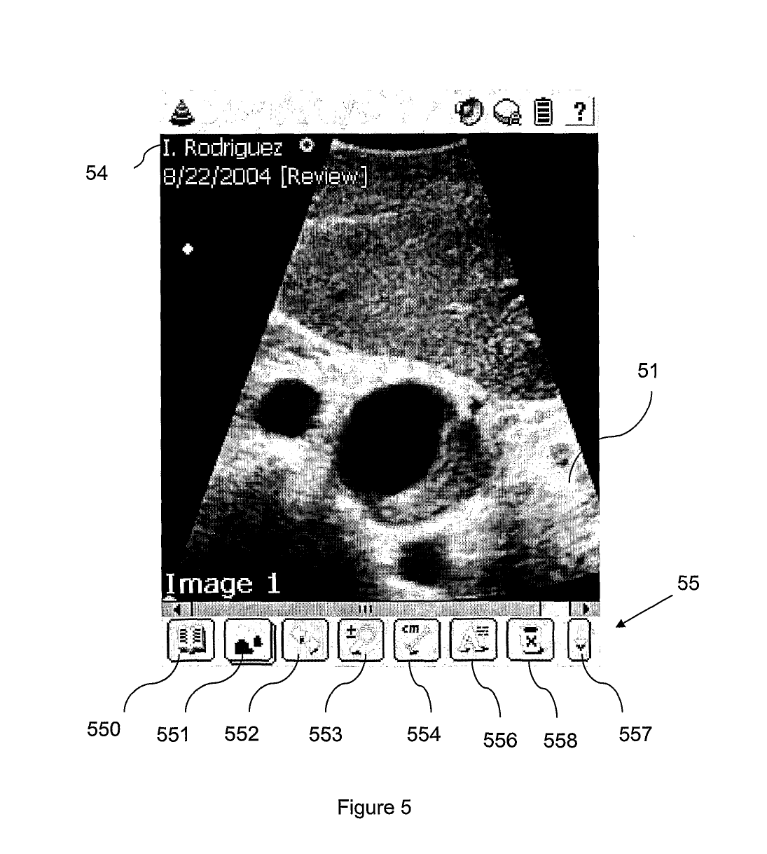

For example, the icon 42 is associated with the ultrasound scan mode. When this is selected, the ultrasound scanning application is run, resulting in the interface display of FIG. 5.

In a preferred embodiment, where the scanner system has only one or a preferred scanning modality, the application associated with that modality, in this case ultrasound scanning, may be run immediately on system start up, with the home screen 40 only being displayed when other functionality or scanning modality is desired to be selected.

This interface display resulting from the running of the ultrasound scanning application includes an area 51 adapted to display the results of the scan from the attached probe unit, in this case, an ultrasound scan. There may also be displayed information pertinent to the type of diagnostic scan being undertaken.

General information which would be relevant to any scan may also be displayed. In this case, the time 53 and the patient name 54 are displayed.

The application also displays a toolbar 55 at the bottom of the display screen. This toolbar includes icons associated with functions relevant to the scan application which is active. For the ultrasound scan application, there is Main Menu icon 550, Exam Management icon 551, Image/Exam Scroller icon 552, Pan/Zoom Control icon 553, Measurement icon 554, and Annotations icon 556. There are also two general function icons which will appear on toolbars associated with more than one application. These are the Delete icon 558 and the Hide Toolbar icon 557.

Icons associated with functions which are not available in the particular context which the application is in, are shown greyed out.

In order to make use of the functions provided by the active application, a user moves the thumbwheel, which highlights each of the icons in turn. When the required function icon is highlighted, the user presses the thumbwheel to select that function, which is then performed by the device.

In embodiments where the display is a touchscreen, the required function may alternatively be selected by touching the screen where the icon is displayed.

In order to make an ultrasound scan, the user, having launched the ultrasound application, presses the Start button 20 to start a scan. A toolbar icon (not shown in the illustrated embodiment) may also be used to start a scan, in a further embodiment, a control may be provided on the probe unit which will also perform this function.

Referring back to FIG. 3, the activation of a scan by a control on the probe unit is detected by the controller 351 and communicated to the DPU 11 via the message channel 310.

Whether initiated by the probe unit control, or by the Start button, at the start of a scan the DPU examines the context and the values of setup parameters to determine if a scan can take place and what settings should be applied for the scan. The DPU then sends a message to the probe unit to initiate a scan which includes any parameters which have been selected for the scan. The controller 351 controls the high voltage driver to produce the required pulse sequence to be applied via the diplexer to the transducer in order to perform a scan according to the parameters set by the user, or set as defaults in the DPU.

The user then rotates the probe as required to sweep the ultrasound beam over the desired area, keeping linear displacement to a minimum, in embodiments where displacement is not sensed.

In embodiments where rotation about all axes is not sensed, the user will also keep rotation about unsensed axes, that is axes about which rotation is not detected by the sensor of the embodiment, to a minimum.

At the same time, data is received from the orientation sensor 19. This is the rotation about the sensed axes of the probe unit. It may be the angular change in the position of the probe unit since the immediately previous transducer pulse, or the orientation of the probe unit in some defined frame of reference. One such frame of reference may be defined by nominating one transducer pulse, normally the first of a scan sequence, as the zero of orientation.

The sensor data and the response signal are passed to the controller 351 where they are combined to give a scanline. A scanline is a dataset which comprises a sequential series of intensity values of the response signal combined with orientation information. A scan dataset is a plurality of sequentially received scanlines.

A scan data set is built up by a user rotating the probe unit about at least one sensed axis while keeping the positional displacement to a minimum. The high voltage generator 312 continues to provide the pulsed voltage to the transducer under control of the microcontroller and each pulse results in a scanline.

Each scanline is a series of values for the intensity of the echo returned from increasing depth into the subject body. The scanline is generated in the controller 351. The scanline data is then passed to a protocol converter to be converted to a protocol suitable for transmission via the data channel. Any suitable protocol may be used. In this embodiment the protocol chosen for use on the data channel is 8b10b, which is well known in the art.

The 8b10b data is passed to a Low Voltage Differential Signal (LVDS) transmitter 338 and is transmitted via the data channel 320 to the DPU 11.

Referring to FIG. 3, the LVDS data channel is received by the DPU via LVDS receiver 321 and phase locked loop 322. The 8b10b data is passed to the DPU processor 340. Protocol conversion is performed by processor 340 to recover the original scanline data.

An application is now run by the DPU processor 340 to process the scanlines for display as an ultrasound image 56 on the display area 51 of the ultrasound application screen.

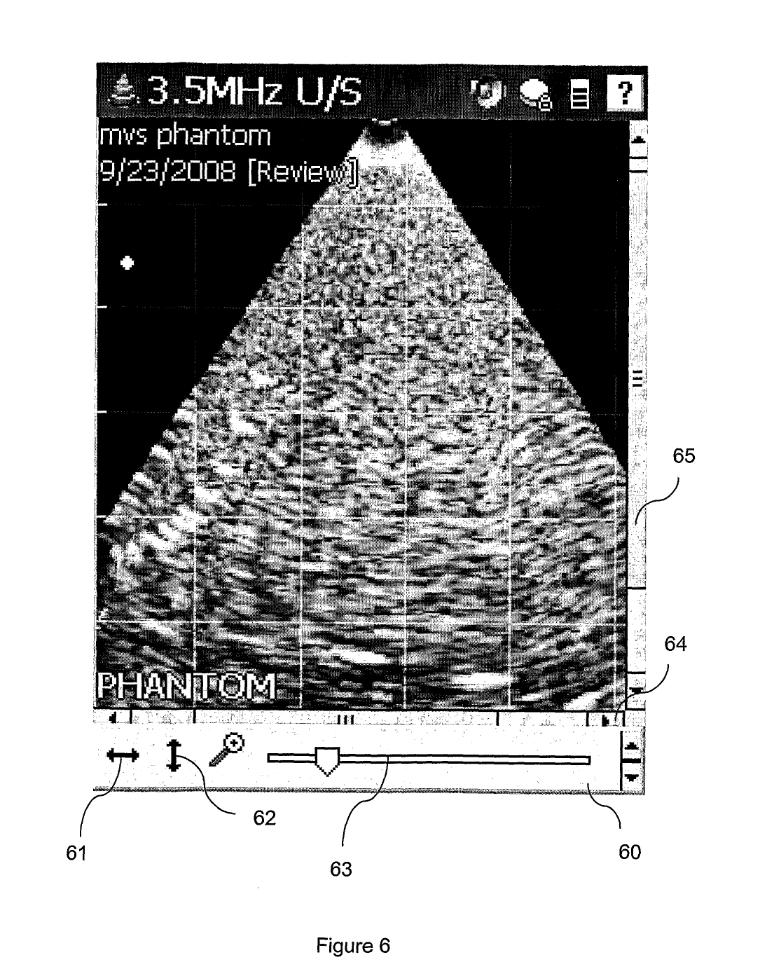

The ultrasound scan image is now available for manipulation by a user. The user may select the Pan/Zoom control 553. This activates the interface screen as illustrated in FIG. 6. There is a sub toolbar 60, which includes control icons being left-right pan control icon 61, up-down pan control icon 62 and zoom control icon 63. The user rotates the thumbwheel to highlight the required control icon, and selects the function by pressing the thumbwheel, or selects that icon using the touch screen. Further rotation of the thumbwheel now controls the application of the control. For example, if the left-right pan control is selected, then rotation of the thumbwheel moves the displayed ultrasound image left to right.

A further press of the thumbwheel causes thumbwheel rotation to return to effecting selection of items on the sub toolbar. Pressing the back button 17, returns thumbwheel control to the toolbar.

In a preferred embodiment with a touchscreen, the zoom may also be controlled by gestures. Moving a finger or stylus against the screen in a circle in one direction, say clockwise causes the image display to zoom such that the image is shown at a greater magnification. Moving a finger or stylus against the screen in a circle in the reverse direction, in this case counter-clockwise causes the image display to zoom such that the image is shown at a lesser magnification.

In preferred embodiments with a touchscreen, left-right slider control 64 and up-down slider bar 65 are also displayed. These may be directly moved by touching the screen to slide the displayed image left to right and up and down.

Measurement of the dimensions of anatomical features shown in an ultrasound image is of great importance in diagnosis and treatment of disease. For example, ultrasound images may show an abdominal aortic aneurysm. (AAA), Such aneurysms of less than about 3 cm in diameter are generally considered to not warrant treatment, while an AAA with a diameter greater than 5 cm would call for immediate medical treatment.

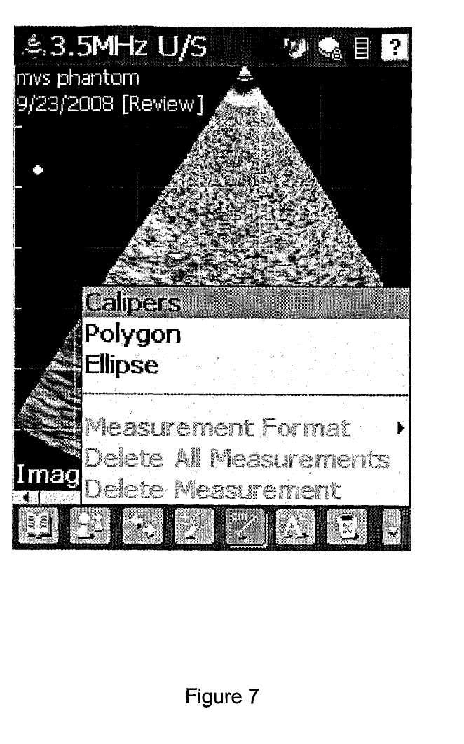

Returning to FIG. 5, the user may select the Measurement control icon 554. This displays a screen shown in FIG. 7. The user may then select, by thumbwheel or stylus, the type of measurement shape that is to be used. The available types are calipers for linear measurement, and polygons and ellipses for area and perimeter measurements.

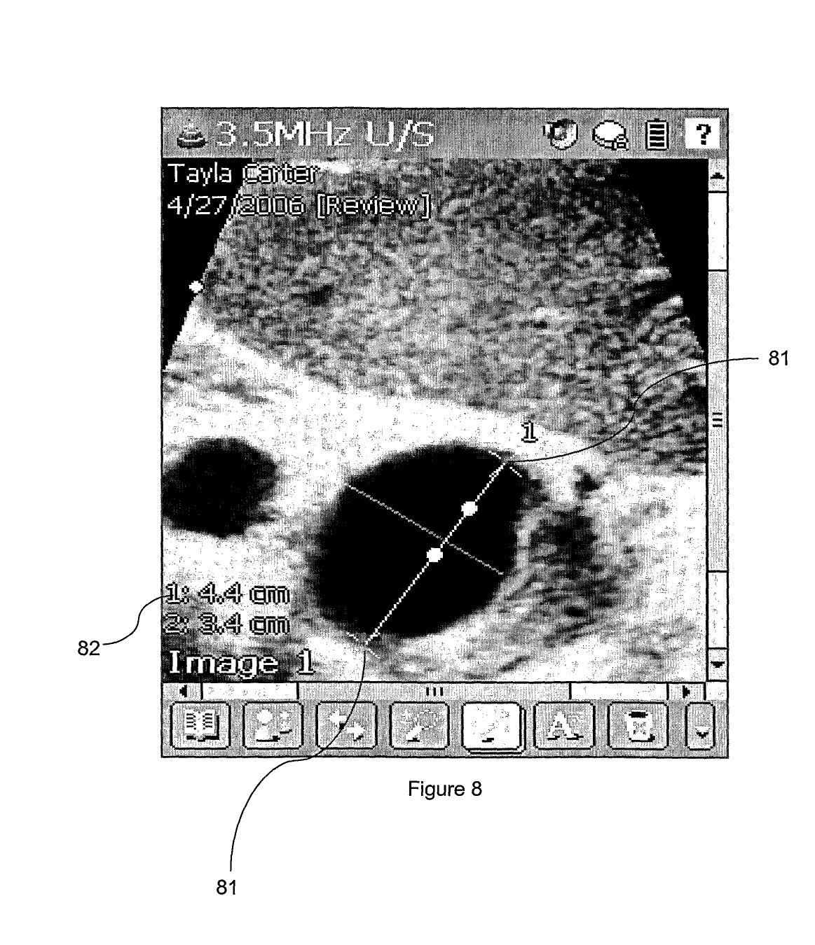

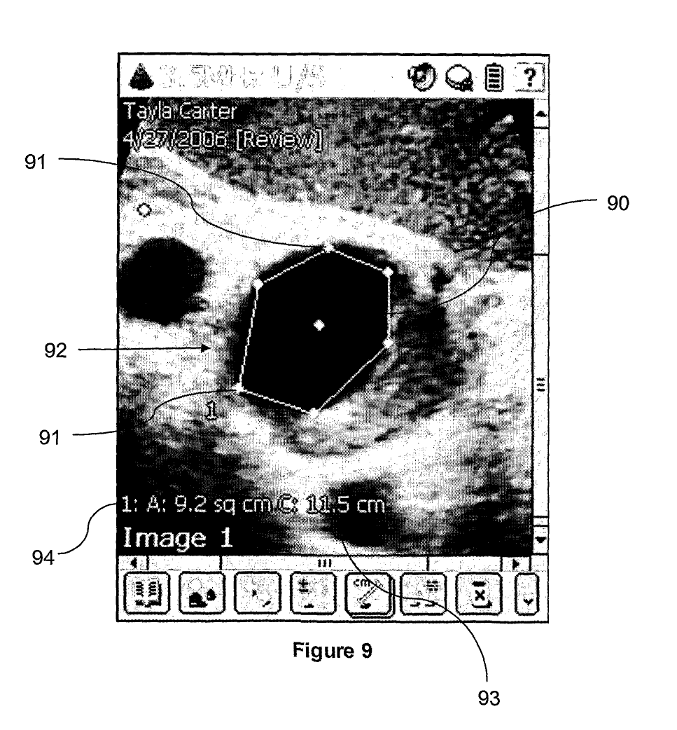

The use of a calliper is illustrated in FIG. 8. The ends 81 of the calliper are moved by dragging with the stylus on a touchscreen until the calliper covers the distance on a feature of the ultrasound image which is to be measured. The measured length 82 is displayed. This length is the real world length of the feature which has been imaged. This calliper may be saved with the image onto storage provided by the system.

Where the perimeter or area of an irregular feature of an image is required, a polygon measurement shape may be applied. This is shown in FIG. 9, where a polygon 90 has been placed on an image. The polygon is initially provided as a square, but additional vertices 91 may be added by tapping a line of the polygon with the stylus. The vertices are moved by a user to cause the polygon to approximately align with the perimeter that is to be measured. In this case the feature 92 is an abdominal aortic aneurysm. The length of the perimeter of the polygon 93 and the area 94 are displayed.

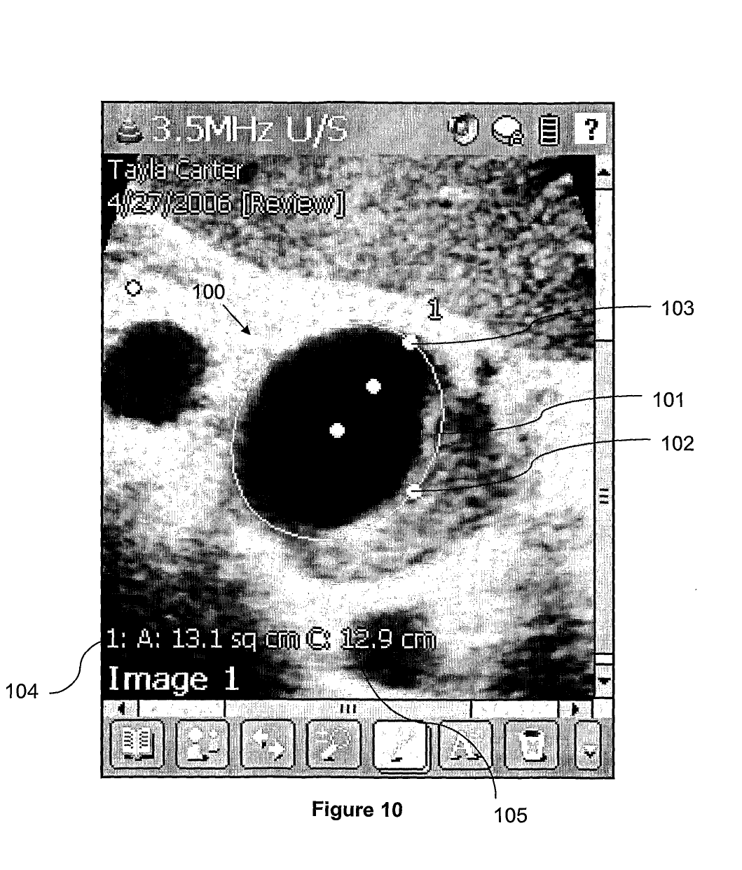

In many cases where anatomy is imaged, features of interest are approximately circular. For ease of measurement of such features an ellipse measure is provided as shown in FIG. 10. There is an ellipsoid feature 100, being the abdominal aorta. A measurement ellipse 101 is displayed over the image. The user drags the measurement ellipse into position over the feature of interest 100. The size and eccentricity of the ellipse are controlled by dragging the drag points 102 and 103 which vary the semimajor and semiminor radii of the ellipse.

The area of the measurement ellipse 104 and its circumference 105 are displayed on the screen.

The information about the dimensions of the anatomical features of the patient gained from the measurement shapes may be used for diagnostic purposes.

The scanner system is able to store images, in a preferred embodiment these are ultrasound scan images of patients.

The images are stored in groups, each of the groups corresponding to a single examination episode, called an exam. This may be the current examination episode, or it may be a previously stored examination episode. The images in each exam will usually be related, as the user seeks to make multiple images of a particular area or organ of interest to assist diagnosis. It is advantageous to be able to scan through these multiple images rapidly to select those which are most relevant.

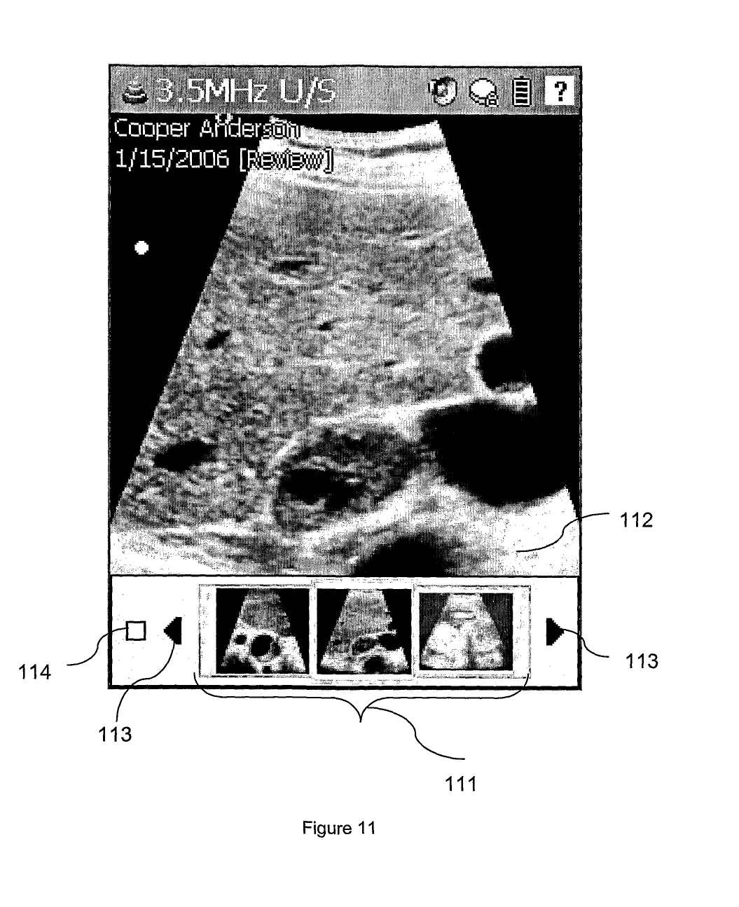

Returning to FIG. 5 a user selects Image/Exam Scroller icon 552, which displays the screen of FIG. 11.

The images within the exam group are shown as thumbnails 111 at the bottom of the display. Three thumbnails are shown at a time. The central thumbnail corresponds to the main displayed image 112. Rotating the thumbwheel or tapping the arrows 113 at each end of the thumbnail display sequentially shows each of the images in the exam group as the corresponding thumbnail is selected.

More than one exam group may be open for display at one time. The Image/Exam Scroller allows scrolling through these exam groups by name to select an exam group whose images are to be displayed.

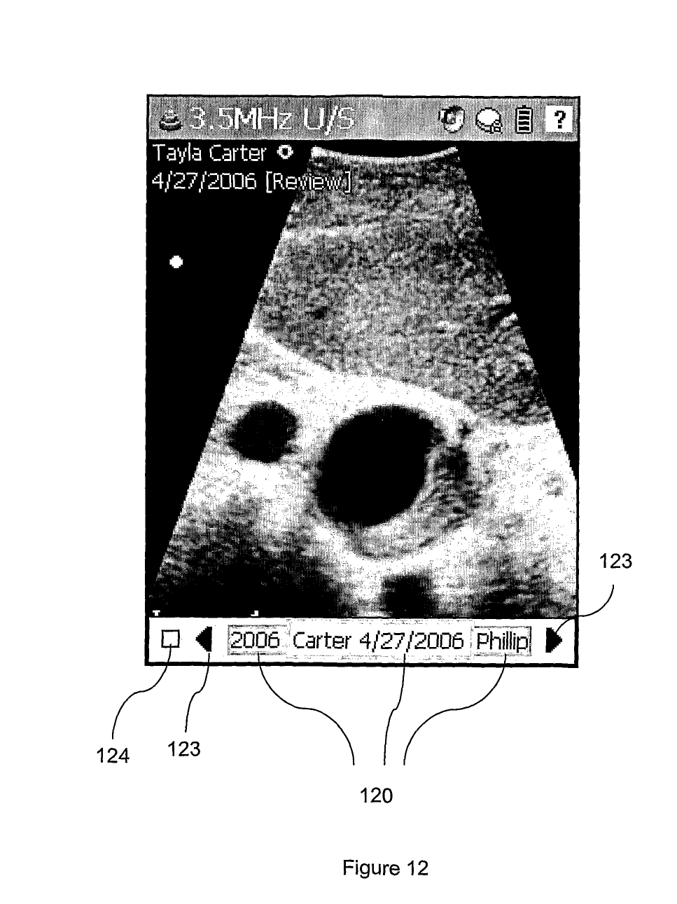

The Exam Scroller function is selected by pressing the thumbwheel, or by tapping the activating icon 114. This results in the display of the screen of FIG. 12.

The available exams 120 are shown identified by an identifying tag, in this case patient name, and a date. The first image of the selected tag group is shown on the main display. The thumbwheel or tapping the arrows 123 allow the user to scroll through the exam groups. Pressing the thumbwheel selects the highlighted exam, the images of which are then shown in the image Scroller, as previously described. The activation icon 124, can also be used to select the exam group by use of the stylus.

It is desirable to be able to associate voice or text annotations with a stored image. Information pertinent to the image can be recorded by a user which will be available when the image is viewed later, or when it is sent to a third party.

Returning to FIG. 5, selecting the Annotations icon 556 pops up window allowing a user to choose to attach a voice or text annotation. A default text annotation, being the description of the scanning mode employed is provided. A series of other likely annotations may also be provided for selection by stylus of thumbwheel scrolling. These may be such tags as “liver”, right kidney”, “aorta” or any other tags likely to be used frequently.

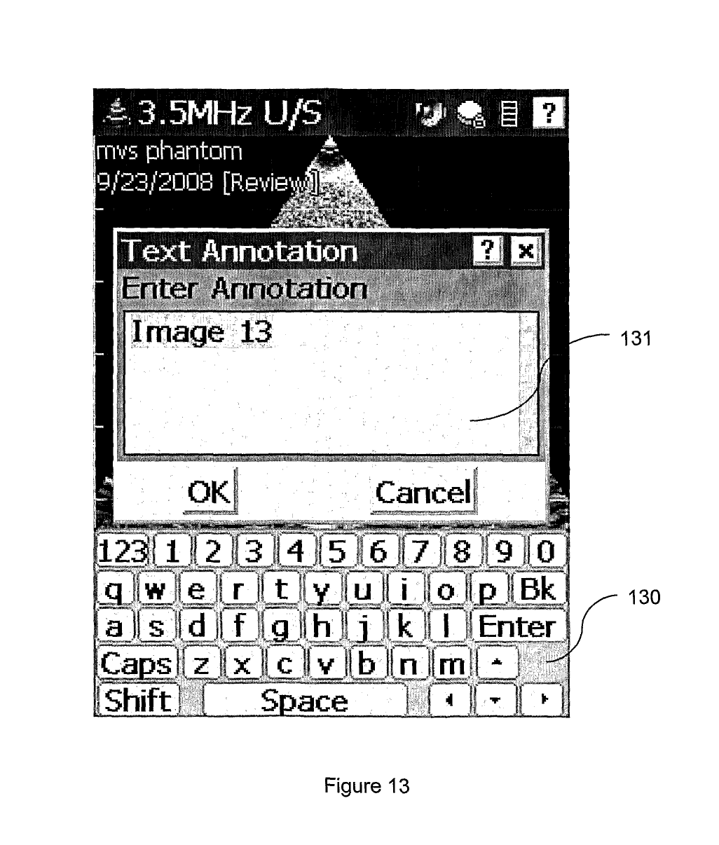

If the user chooses to add a different text annotation, the screen of FIG. 13 is displayed. There is a soft keyboard 130 and a text entry area 131. The user uses the stylus to choose characters from the soft keyboard to make up an annotation, or the user may use the thumbwheel to scroll through the letters of the soft keyboard, pressing the thumbwheel to select the desired character for insertion into the text area. The text annotation is then displayed on the image, and saved to memory as part of the stored image.

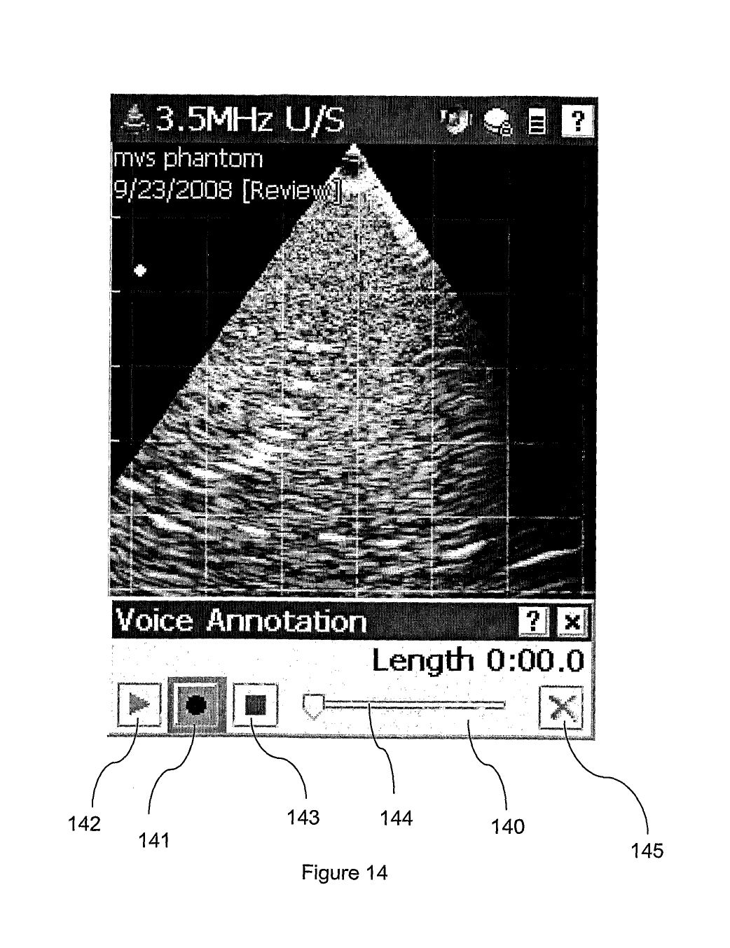

Choosing to add a voice annotation from the pop up screen, gives the screen of FIG. 14. A voice annotation pop up window 140 is displayed. This window has icons for Record 141, Play 142 and Stop 143. It also has an indication of the position within the recording 144. The length of the recoding is also provided.

Using the stylus or the thumbwheel, the user selects the Record icon 141, then speaks the words to be the annotation. At the end of the annotation the user selects the Stop icon 143. The speech is recorded, and saved with the displayed image. The voice annotation may be played by selecting the Play icon 142.

There is also provide a delete icon 145, which, when selected, causes the annotation to be deleted.

The above usage of the interface is an exemplary embodiment. Control of any function of a hand-held medical diagnostic device may be implemented by the interface. The required function is broken down into a series of procedures. Each procedure may contain a group of sub-procedures, and each of those sub-procedures may contain further group of sub-procedures, without limit, except the practical limits of the processing apparatus.

In an embodiment each procedure is designed such that it may be controlled by a control element requiring only the three available control movements provided by a thumbwheel, or similar single handed control member, that is, forward rotation, reverse rotation and pressing. In addition, a further control buttons which act to activate the immediately previously selected control level or to act as a “start” button may be provided.

At the highest level, each function is represented by an icon on a toolbar, displayed on a screen. The rotation of the thumbwheel highlights each icon on the toolbar, and pressing the thumbwheel selects the function.

The first procedure of the function is launched. This may perform an operation directly. For more complex procedures, it may display one or more controls. These controls are operated by the thumbwheel to perform such actions as panning and zooming an image, moving tags or selecting menu items from displayed menus.

For yet more complex procedures, a further toolbar of option icons may be displayed. Again the rotation of the thumbwheel highlights each icon on the toolbar in turn, and pressing the thumbwheel selects the option. Again there are the three possibilities of directly performing an action, displaying a control, or displaying a further toolbar. This sequence may repeat until all of the procedures of the function as required by the user have been carried out. Pressing the back button will always result in the immediately previously active toolbar being displayed.

Where text input is required, a text input area and a soft keyboard will be displayed on the display screen. Rotation of the thumbwheel is used to highlight the required character, and the thumbwheel is pressed to select the highlighted character. In a preferred embodiment, a touchscreen and stylus are provided to allow the user to select characters from the soft keyboard.

Text menus of options and dialog boxes for setting option values may be accessed in the same way.

The applications which are available depend upon the nature of the probe unit. Probe units define the functions available to the scanner device. For example, a probe unit adapted to act as an ECG scanner would require the DPU to run a program with functions appropriate to such a scan. The basic interface with icon toolbars, direct action icons and controls can be adapted to provide controls for any hand-held scanning device.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-13. (canceled)

14. A hand held medical diagnostic scanner system having a user interface including:
a. a display screen,
b. a control member configured to:
(1) sense a first user control movement oriented in at least one direction, and
(2) communicate the first user control movement to software configured to translate this movement into selection of an option indicator from option indicators displayed on the display screen,
(3) sense a second user control movement different from the first user control movement, and
(4) communicate the second user control movement to software configured to translate this movement into the activation of an option associated with a selected option indicator.

15. The scanner system of claim 14 wherein the control member includes at least one of:
a. a depressable thumbwheel, and
b. a touch-sensitive panel.

16. The scanner system of claim 14 wherein:
a. the control member is a thumbwheel protruding from a casing,
b. the first user control movement includes rotating the thumbwheel about an axis,
c. the second user control movement includes depressing the thumbwheel toward the casing.

17. The scanner system of claim 16 wherein the display screen is a touch screen.

18. The scanner system of claim 16 wherein the option indicators are icons, with each icon being associated with a different function of the scanner system.

19. The scanner system of claim 18 wherein the display screen concurrently displays:
a. at least a portion of a scanned body, and
b. several icons, each icon being associated with:
(1) a function related to the displayed scanned body or portion thereof, or
(2) the setup of the scanner system.

20. The scanner system of claim 18 wherein the scanner system is an ultrasound scanner system.

21. The scanner system of claim 14 wherein:
a. the control member is a touch sensitive panel,
b. the first user control movement includes sliding a user's digit along the panel, and
c. the second user control movement includes tapping the panel.

22. The scanner system of claim 21 wherein the touch sensitive panel is part of the display screen.

23. A hand held medical diagnostic scanner system including:
a. an interface displaying a toolbar having icons thereon, each icon representing a function to be controlled by the interface,
a. a control member configured for single-handed use, the control member further being configured to produce:
(1) a directional output, the directional output providing user selection of an icon,
(2) a selection output, the selection output:
(a) performing the function represented by the selected icon, or
(b) displaying a further toolbar displaying further icons, each icon representing a procedure or control associated with the function represented by the selected icon, wherein the directional output of the control member provides:
i. user selection of one of the further icons to activate the procedure represented by the further icon, or
ii. user movement of one of the further icons to adjust the control represented by the icon,
wherein at least most of the functions of the interface are controllable solely by user input from the control member.

24. The user interface of claim 23 further including a second control member:
a. configured for single handed use, and
b. configured to produce a selection output, the selection output resulting in a display of a previously displayed toolbar.

25. The user interface of claim 24 wherein:
a. the control member is a thumbwheel, and
b. the second control member is a button.

26. A hand-held medical diagnostic scanner system including:
a. a ultrasonic probe having a size and weight such that it can readily be grasped, supported, and repositioned by a single adult user's hand;
b. a hand-held display unit:
(1) having a size and weight such that it can readily be grasped, supported, and repositioned by a single adult user's hand,
(2) receiving measurements from the probe, and
(3) including:
(a) a display screen having option indicators displayed thereon,
(b) a control member configured to receive:
i. a first user control input movement oriented in at least one direction, and
ii. a second user control input movement different from the first user control input movement, and
wherein the display unit:
I. converts the first user control input movement into selection of one of the option indicators displayed on the display screen,
II. converts the second user control input movement into the activation of an function associated with the selected option indicator.

27. The scanner system of claim 26 wherein the option indicators are icons, with each icon being associated with a different function of the scanner system.

28. The scanner system of claim 26 wherein the display screen concurrently displays:
a. at least a portion of an ultrasound image generated from measurements from the probe, and
b. an option indicator allowing manipulation of the displayed ultrasound image or portion thereof.

29. The scanner system of claim 26 wherein:
a. the display screen is situated adjacent a display unit casing,
b. the control member is a wheel protruding from the display unit casing,
c. the first user control input movement includes rotating the thumbwheel,
d. the second user control input movement includes depressing the thumbwheel.

30. The scanner system of claim 29 wherein the display screen is touch-sensitive, and accepts user control input movements including:
a. sliding an item in contact with the display screen along the display screen, and
b. tapping the display screen.

31. The scanner system of claim 26 wherein:
a. the control member is a touch sensitive panel,
b. the first user control movement includes sliding an item in contact with the panel along the panel, and
c. the second user control movement includes tapping the panel.

32. The scanner system of claim 31 wherein the panel is at least partially defined by the display screen.

33. The scanner system of claim 26 wherein:
a. a cable extends between the probe and the display unit,
b. the probe is connected solely to the cable, and the display unit is also solely connected to the cable, such that the probe and display unit define opposing terminal ends of the scanner system.
